(19) United States
(12) Patent Application Publication
(10) Pub. No.: US 2004/0044323 A1
(43) Pub. Date: Mar. 4, 2004

(54) Absorbent article with elastic components having non-uniform elastic tension

(75) Inventors: Thomas H. Roessler, Menasha, WI (US); Emily J. Baum, Appleton, WI (US); Roman A. Weyenberg JR., Neenah, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/231,535

(22) Filed: Aug. 30, 2002

(51) Int. Cl. A61F 13/15; A61F 13/20

(52) U.S. Cl. 604/385.24

(57) ABSTRACT

An absorbent article has an anterior region, a posterior region and a central region interconnecting the anterior and posterior regions and extending longitudinally therebetween. An elastic component extends generally continuously along a path from the anterior region of the article through the central region to the posterior region thereof. The elastic component has an elastic tension which is substantially non-uniform along the path defined by the elastic component. In one method for making such an absorbent article, an elastic component is initially secured to the article along the path and defines a width thereof. At least a portion of the elastic component is then removed from the article to narrow the width of the path defined by the elastic component along at least a segment of the path to thereby provide an elastic tension gradient along the length of the path.

### BACKGROUND OF THE INVENTION

The present invention relates generally to absorbent articles, such as those used as personal care products, and more particularly to such an absorbent article having variable tension elastic components for improved fit.

Disposable absorbent articles find widespread use as personal care products such as diapers, children's toilet training pants, adult incontinence garments, medical garments, sanitary napkins and the like, as well as surgical bandages and sponges. These articles absorb and contain body waste and are typically disposable in the sense that they are intended to be discarded after a limited period of use; i.e., the articles are not intended to be laundered or otherwise restored for reuse. Conventional disposable absorbent articles comprise an absorbent body disposed between a liner adapted for contiguous relationship with the wearer's skin and an outer cover for inhibiting liquid body waste absorbed by the absorbent body from leaking out of the article. The liner of the absorbent article is typically liquid permeable to permit liquid body waste to pass therethrough for absorption by the absorbent body.

To improve the fit of disposable absorbent articles on the wearer, it is known to incorporate elastic components, such as elastic strands or threads, into the article. For example, diapers, training pants, adult incontinence garments or other pants-like articles may secure leg elastic components within the article, e.g., at the leg openings thereof, while the elastic components are in a stretched condition so that the retractive forces of the elastic components gather the article at the leg openings to provide a snug fit around the wearer's leg.

Leg elastic components of conventional diapers typically provide an elastic tension which is substantially uniform along the lengths of the elastic components before the diaper is worn. When securing the diaper on a baby, the baby's legs are first spread apart almost at a 90° angle to the baby's torso. The leg elastic components thus generally land in the mid thigh region of the baby. However, when the baby stands, or sits, the baby's legs come together which forces the leg elastic components to conform to the creases formed between the baby's legs and torso. As a result, the elastic components in the crotch and front region of the diaper elongate inward to conform to the baby's body lines, thus increasing the elastic tension of the elastic components in those regions. The baby's back leg region does not significantly change shape following securement of the diaper on the baby and, as such, the elastic tension in the back region of the diaper does not change considerably when the baby stands or sits.

It is known that increased elastic tension in the front and crotch regions of the diaper can cause red-marking (e.g., indentations) in the baby's skin during wear. One conventional approach to reducing red-marking is to reduce the elastic tension along the entire length of the leg elastic, thereby reducing the elastic tension in the front and crotch regions of the diaper. However, the reduced elastic tension along the length of the leg elastic may result in a loose fit and possibly even leakage around the back leg region of the diaper. There is a need, therefore, for a disposable absorbent article, and more particularly a diaper, having leg elastic components in which the initial elastic tension of the elastic components is lower in the front and crotch regions of the diaper than in the back region of the diaper so that the applied elastic tension is more uniform during wear.

Leg elastic components of conventional disposable absorbent articles such as diapers are also typically positioned laterally inward of the respective laterally opposite side edges of the diaper, particularly at the leg openings of the diaper. Thus, ungathered material of the diaper extends laterally outward of the leg elastic components and has a tendency to fold inward as the diaper is being secured to the baby, thereby reducing the back leg coverage by the diaper. The person securing the diaper to the baby then has to reach into the diaper and pull the ungathered region out, which is an additional step that reduces the convenience of the diaper.

### SUMMARY OF THE INVENTION

In general, one embodiment of an absorbent article of the present invention comprises an anterior region, a posterior region and a central region interconnecting the anterior and posterior regions and extending longitudinally therebetween. An elastic component extends generally continuously along a path from the anterior region of the article through the central region to the posterior region thereof. The elastic component has an elastic tension which is substantially non-uniform along the path defined by the elastic component.

In another embodiment, an absorbent article has a longitudinal axis and comprises an anterior region, a posterior region and a central region interconnecting the anterior and posterior regions and extending longitudinally therebetween. An elastic component extends generally continuously along a path from the anterior region of the article through the central region to the posterior region thereof. The elastic component defines an outer boundary of the path. At least a portion of the outer boundary extends generally non-parallel to the longitudinal axis of the article and defines an outer curvature of the elastic component which is greater than one inch.

One embodiment of a method of the present invention for making an absorbent article having an anterior region, a posterior region and a central region interconnecting the anterior and posterior regions and extending longitudinally therebetween generally comprises securing an elastic component to the absorbent article along a path which extends generally continuously from the anterior region of the article through the central region to the posterior region thereof. The elastic component defines a width of the path. At least a portion of the elastic component of the article is removed to narrow the width of the path defined by the elastic component along at least a segment of the path to provide an elastic tension gradient along the length of the path.

### BRIEF DESCRIPTION OF THE DRAWINGS

FIG. 1 is a plan view of an absorbent article of the present invention illustrated in the form of a diaper shown unfastened and laid flat and with portions of the diaper broken away to reveal internal construction thereof;

FIG. 2 is a fragmentary cross section generally taken in the plane including line 2-2 of FIG. 1; and

FIG. 3 is a perspective view of the diaper shown as worn.

Corresponding reference characters indicate corresponding parts throughout the several views of the drawings.

### DEFINITIONS

Within the context of this specification, each term or phrase below will include the following meaning or meanings:

(a) “Bonded” refers to the joining, adhering, connecting, attaching, or the like, of two elements. Two elements will be considered to be bonded together when they are bonded directly to one another or indirectly to one another, such as when each is directly bonded to intermediate elements.

(b) “Film” refers to a thermoplastic film made using a film extrusion and/or foaming process, such as a cast film or blown film extrusion process. The term includes apertured films, slit films, and other porous films which constitute liquid transfer films, as well as films which do not transfer liquid.

(c) “Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

(d) “Layer” when used in the singular can have the dual meaning of a single element or a plurality of elements. (e) “Liquid impermeable,” when used in describing a layer or multi-layer laminate means that liquid body waste, such as urine, will not pass through the layer or laminate, under ordinary use conditions, in a direction generally perpendicular to the plane of the layer or laminate at the point of liquid contact.

(f) “Liquid permeable” refers to any material that is not liquid impermeable.

(g) “Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are preferably substantially continuous in length.

(h) “Non-woven” and “non-woven web” refer to materials and webs of material which are formed without the aid of a textile weaving or knitting process.

(i) “Pliable” refers to materials which are compliant and which will readily conform to the general shape and contours of the wearer's body.

(j) “Spunbond” refers to small diameter fibers which are formed by extruding molten thermoplastic material as filaments from a plurality of fine capillaries of a spinnerette having a circular or other configuration, with the diameter of the extruded filaments then being rapidly reduced by a conventional process such as that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, more particularly, between about 0.6 and about 10.

(k) “Superabsorbent” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

(l) “Thermoplastic” describes a material which softens when exposed to heat and which substantially returns to a non-softened condition when cooled to room temperature.

(m) “Stretchable”, refers to materials which are either elastic or extensible, that is materials which when elongated in one or more dimensions either exert a force tending to move the material at least partially to its original dimensions (elastic), or which remain in the elongated configuration (extensible).

### DETAILED DESCRIPTION OF THE INVENTION

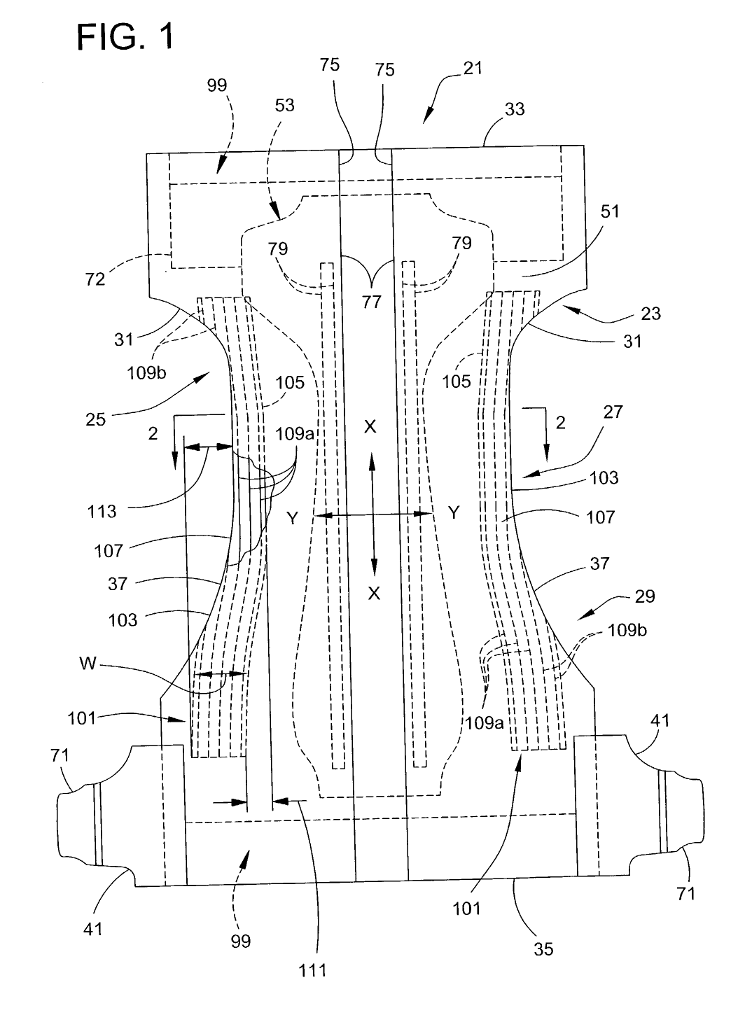

Referring now to the drawings and in particular to FIG. 1, an absorbent article constructed in accordance with the present invention is illustrated in the form of a diaper, which is indicated in its entirety by the reference numeral 21. As used herein, an absorbent article refers to an article which may be placed against or in proximity to the body of the wearer (e.g., contiguous to the body) to absorb and/or retain various waste discharged from the body. Some absorbent articles, such as disposable absorbent articles are intended to be discarded after a limited period of use instead of being laundered or otherwise restored for reuse. It is contemplated, however, that the principles of the present invention have application in garments (including reusable garments) and other absorbent articles. For example, the principles of the present invention may be incorporated into children's training pants and other infant and child care products, adult incontinence garments and other adult care products, medical garments, sanitary napkins and other feminine care products and the like, as well as surgical bandages and sponges.

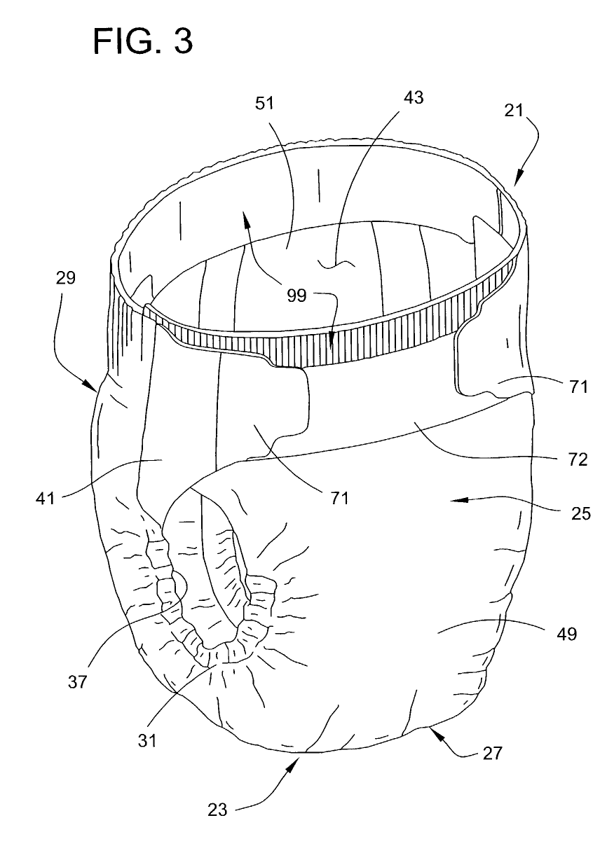

The diaper 21 is shown in FIG. 1 in an unfolded and laid-flat condition to illustrate a longitudinal axis X and a lateral axis Y of the diaper. The diaper 21 generally comprises a central absorbent assembly 23 extending longitudinally from a front (e.g., anterior) region 25 of the diaper through a crotch (e.g., central) region 27 to a back (e.g., posterior) region 29 of the diaper. The central absorbent assembly 23 is generally I-shaped, and more particularly hourglass shaped, and has contoured, laterally opposite side edges 31 and longitudinally opposite front and rear waist edges or ends, respectively designated 33 and 35. It is understood, however, that the diaper 21 may have other shapes, such as a rectangular shape or a T-shape without departing from the scope of the present invention. The side edges 31 of the diaper 21 extend longitudinally from the front region 25 through the crotch region 27 to the back region 29 for forming transversely spaced leg openings 37 (FIG. 3) of the diaper when worn.

The diaper 21 is refastenably secured on a wearer's body using suitable fastening components as described later herein to refastenably secure the back region 29 to the front region 25 of the diaper. The front region 25 generally includes the portions of the central absorbent assembly 23 which extend over the wearer's lower abdominal region and the back region 29 generally includes the portions of the central absorbent assembly which extend over the wearer's lower back region. The crotch region 27 includes the portion extending longitudinally through the wearer's crotch from the front region 25 to the back region 29 and laterally between the wearer's legs. As worn on the wearer's body (FIG. 3), the diaper 21 further defines a central waist opening 43 and the leg openings 37.

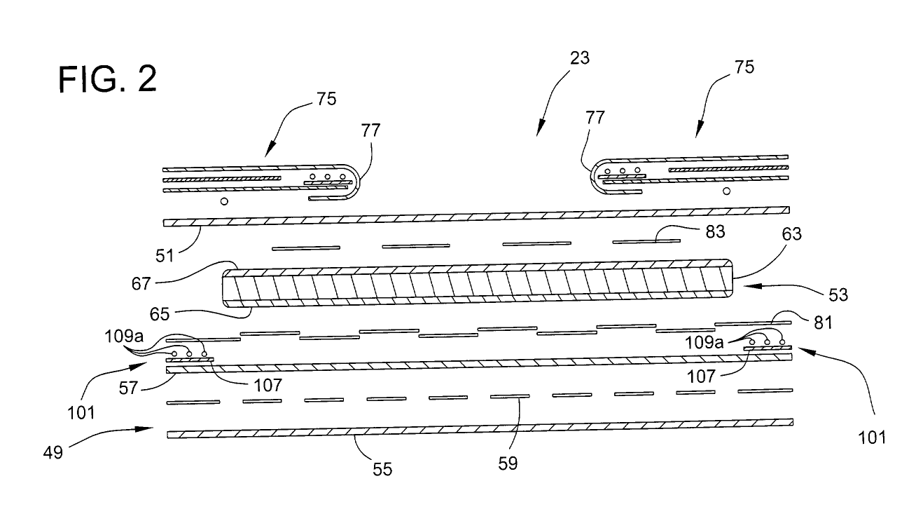

With particular reference to FIG. 2, the central absorbent assembly 23 of the diaper 21 comprises an outer cover, generally indicated at 49, a bodyside liner 51 positioned in facing relation with the outer cover, and an absorbent body 53, generally indicated at 53, disposed between the outer cover and the liner. The outer cover 49 of the illustrated embodiment generally defines the length and width of the diaper 21. The absorbent body 53 has a length and width which are less than the length and width of the outer cover 49 such that the outer cover extends both longitudinally and laterally out beyond the sides and ends of the absorbent body. The bodyside liner 51 may be generally coextensive with the outer cover 49, or may instead overlie an area which is larger (and would thus generally define the length and/or width of the diaper 21) or smaller than the area of the outer cover 49, as desired. In other words, the bodyside liner 51 is preferably in superposed relation with the outer cover 49 but may not necessarily be coextensive with the outer cover.

In one embodiment, the outer cover 49 is stretchable and may or may not be somewhat elastic. More particularly, the outer cover 49 is extensible such that once stretched under the weight of the insulted absorbent body, the outer cover will not retract substantially back toward its original position. For example, the outer cover 49 may be stretched approximately 25% to 150% beyond its original length with a relatively low force required to extend. More desirably, the outer cover 49 may be stretched approximately 50% to 100% beyond its original length and most preferably about 50% beyond its original length under a low stretching force. As a further example, in one embodiment a 25% elongation is achieved upon application of a force of in the range of about 30 g/in to about 200 g/in, more preferably between about 70 g/in and 150 g/in and most preferably about 100 g/in. It is also contemplated that the outer cover 49 may instead be generally non-extensible and remain within the scope of this invention.

The outer cover 49 is also desirably constructed to support a selected hydrohead of water substantially without leakage therethrough. A suitable technique for determining the resistance of a material to liquid penetration is Federal Test Method Standard FTMS 191 Method 5514, 1978, or an equivalent thereof. Since the outer cover 49 is extensible, a layer of nylon net material having a thickness of about 0.1 mm may be needed to support the outer cover material for this test. The net material may be provided by nylon threads arranged in a hexagonal or honeycomb-like pattern with openings approximately 4 mm across. For example, the net material may be purchased from Wal-Mart Stores under the trade designation T-246. The net material is liquid pervious and does not significantly affect the hydrohead values obtained. The extensible outer cover 49 is desirably sufficiently impermeable to liquid and semi-liquid materials to substantially prevent the undesired leakage of waste materials, such as urine and feces. For example, the extensible outer cover 49 can desirably support a hydrohead of at least about 45 centimeters (cm) substantially without leakage. The extensible outer cover 49 can alternatively support a hydrohead of at least about 55 cm, and optionally, can support a hydrohead of at least about 60 cm, or more, to provide improved benefits.

The extensible outer cover 49 can be composed of various materials which provide the desired properties set forth herein. For example, the extensible outer cover 49 is desirably composed of a neckable or otherwise necked fabric, but may instead, or may additionally, be composed of a creped fabric, a crimped fiber fabric, an extendable fiber fabric, a bonded-carded fabric, a micro-pleated fabric, polymer films or the like. The fabrics may be woven or nonwoven materials, such as spunbond fabrics.

As used herein, the term “neck” or “neck stretch” interchangeably means that a material is drawn such that it is extended under conditions reducing its width or its transverse dimension by drawing and elongating to increase the length of the fabric. The controlled drawing may take place under cool temperatures, room temperature or greater temperatures and is limited to an increase in overall dimension in the direction being drawn up to the elongation required to break the fabric. The necking process typically involves unwinding a sheet from a supply roll and passing it through a brake nip roll assembly driven at a given linear speed. A take-up roll or nip, operating at a linear speed higher than the brake nip roll, draws the fabric and generates the tension needed to elongate and neck the fabric. U.S. Pat. No. 4,965,122 entitled REVERSIBLY NECKED MATERIAL, by M. T. Morman which issued Oct. 23, 1990, the entire disclosure of which is hereby incorporated by reference, discloses a process for providing a reversibly necked non-woven material which may include necking the material, then heating the necked material, followed by cooling.

As used herein, the term “neckable material or layer” means any material which can be necked such as a nonwoven, woven, or knitted material. The term “necked material” refers to any material which has been drawn in at least one dimension, (e.g. lengthwise), reducing the transverse dimension, (e.g. width), such that when the drawing force is removed, the material can be pulled back to its original width. The necked material typically has a higher basis weight per unit area than the un-necked material. When the necked material is pulled back to its original un-necked width, it should have about the same basis weight as the un-necked material. This differs from stretching/orienting a material layer, during which the layer is thinned and the basis weight is permanently reduced.

Typically, such necked nonwoven fabric materials are capable of being necked up to about 80 percent. For example, the extensible outer cover 49 may be composed of a material which has been necked from about 10 to about 80 percent, desirably from about 20 to about 60 percent, and more desirably from about 30 to about 50 percent for improved performance. For the purposes of the present disclosure, the term “percent necked” or “percent neckdown” refers to a ratio or percentage determined by measuring the difference between the pre-necked dimension and the necked dimension of a neckable material, and then dividing that difference by the pre-necked dimension of the neckable material and multiplying by 100 for percentage. The percent necked can be determined in accordance with the description in the above-mentioned U.S. Pat. No. 4,965,122.

The outer cover 49 may be a multi-layered laminate structure, and more desirably a necked, multi-layer laminate structure, to provide desired levels of extensibility as well as liquid impermeability and vapor permeability. For example, the outer cover 49 of the illustrated embodiment is of two-layer construction, including an outer layer 55 constructed of a vapor and liquid permeable necked material and an inner layer 57 constructed of a liquid impermeable material, with the two layers being secured together by a suitable laminate adhesive 59. It is understood, however, that the outer cover 49 may instead be constructed of a single layer of liquid impermeable material, such as a thin plastic film constructed of materials such as those from which the inner layer 57 is constructed as described later herein, without departing from the scope of this invention.

The liquid permeable outer layer 55 can be any suitable material as described above and is desirably one which provides a generally cloth-like texture. Suitable neckable materials for the outer layer 55 include non-woven webs, woven materials and knitted materials such as those described in the above-mentioned U.S. Pat. No. 4,965,122. Non-woven fabrics or webs have been formed from many processes, for example, bonded carded web processes, meltblowing processes and spunbonding processes. The non-elastic neckable material is preferably formed from at least one member selected from fibers and filaments of inelastic polymers. Such polymers include polyesters, for example, polyethylene terephthalate, polyolefins, for example, polyethylene and polypropylene, polyamides, for example, nylon 6 and nylon 66. These fibers or filaments are used alone or in a mixture of two or more thereof. Suitable fibers for forming the neckable material include natural and synthetic fibers as well as bicomponent, multi-component, and shaped polymer fibers.

Many polyolefins are available for fiber production according to the present invention, for example, fiber forming polypropylenes include Exxon Chemical Company's Escorene PD 3445 polypropylene and Himont Chemical Company's PF-304. Polyethylenes such as Dow Chemical's ASPUN 6811A linear low density polyethylene, 2553 LLDPE and 25355 and 12350 high density polyethylene are also suitable polymers. The nonwoven web layer may be bonded to impart a discrete bond pattern with a prescribed bond surface area. If too much bond area is present on the neckable material, it will break before it necks. If there is not enough bond area, then the neckable material will pull apart. Typically, the percent bonding area useful in the present invention ranges from around 5 percent to around 40 percent of the area of the neckable material.

One particular example of suitable material from which the outer layer 55 may be constructed is a 0.4 osy (ounce per square yard) or 14 gsm (grams per square meter) spunbond polypropylene non-woven web which is neckable in the range of about 35% to 45%. The outer layer 55 may also be constructed of the same materials from which the bodyside liner 51 is constructed as described later herein. Also, while it is not a necessity for the outer layer 55 of the outer cover 49 to be liquid permeable, it is desired that it have a cloth-like texture.

The liquid impermeable inner layer 57 of the outer cover 49 can be either vapor permeable (i.e., “breathable”) or vapor impermeable. The inner layer 57 is desirably manufactured from a thin plastic film, although other flexible liquid impermeable materials may also be used. More particularly, the inner layer 57 can be made from either cast or blown film equipment, can be coextruded and can be embossed if so desired. It is understood that the inner layer 57 may otherwise be made from any suitable non-elastic polymer composition and may include multiple layers. Where the inner layer 57 is vapor permeable, it may contain such fillers as micropore developing fillers, e.g. calcium carbonate; opacifying agents, e.g. titanium dioxide; and antiblock additives, e.g. diatomaceous earth. Suitable polymers for the inner layer 57 include but are not limited to non-elastic extrudable polymers such as polyolefin or a blend of polyolefins, nylon, polyester and ethylene vinyl alcohol. More particularly, useful polyolefins include polypropylene and polyethylene. Other useful polymers include those described in U.S. Pat. No. 4,777,073 to Sheth, assigned to Exxon Chemical Patents Inc., such as a copolymer of polypropylene and low density polyethylene or linear low density polyethylene.

Alternative polymers for the inner layer 57 include those referred to as single site catalyzed polymers such as “metallocene” polymers produced according to a metallocene process and which have limited elastic properties. The term “metallocene-catalyzed polymers” as used herein includes those polymer materials that are produced by the polymerization of at least ethylene using metallocenes or constrained geometry catalysts, a class of organometallic complexes, as catalysts. For example, a common metallocene is ferrocene, a complex of a metal between two cyclopentadienyl (Cp) ligands. Such metallocene polymers are available from Exxon Chemical Company of Baytown, Tex. under the trade name EXXPOL® for polypropylene based polymers and EXACT® for polyethylene based polymers and from Dow Chemical Company of Midland, Mich. under the name ENGAGE®. Preferably, the metallocene polymers are selected from copolymers of ethylene and 1-butene, copolymers of ethylene and 1-hexene, copolymers of ethylene and 1-octene and combinations thereof.

The inner layer 57 may be laminated to the neckable material of the outer layer 55 to form the laminate outer cover 49 by conventional methods known in the art including adhesive bonding, point bonding, thermal point bonding, and sonic welding. The outer cover 49 is then necked by conventional necking processes which typically vary the surface speed of the web to draw or neck the laminate. Such necking provides striated rugosities in the film and/or laminate resulting in transverse extensibility to the necked laminate and more “cloth-like” aesthetics. It is known that stretching and orienting a filled film layer (e.g., inner layer 57) causes micropores to form in the film, but longitudinal striated rugosities do not typically form in the film layer when stretched. The film layer would instead become physically thinner and may narrow slightly. By necking the laminate, the non-elastic neckable material, which is attached to the non-elastic film layer, will neck and bring the non-elastic film layer with it, thereby forming the longitudinal striated rugosities in the film which allow the film layer to extend in the transverse direction.

Alternative necked laminate materials that could be used to provide the outer cover 49 with the desired extensibility and liquid impermeability are described in U.S. patent application Ser. No. 09/460,490 filed Dec. 14, 1999 and entitled “BREATHABLE LAMINATE PERMANENTLY CONFORMABLE TO THE CONTOURS OF A WEARER”, the entire disclosure of which is hereby incorporated by reference. Other suitable necked laminates that include at least one non-elastic neckable material laminated to at least one non-elastic film material are described in U.S. patent application Ser. No. 09/455,513 filed Dec. 6, 1999 and entitled “TRANSVERSELY EXTENSIBLE AND RETRACTABLE NECKED LAMINATE OF NON-ELASTIC SHEET LAYERS”, the entire disclosure of which is hereby incorporated by reference. However, it is to be understood that the laminate outer cover need not be composed of a neckable or necked material to remain within the scope of this invention.

In an another embodiment, the outer cover 49 of the diaper 21 may instead be substantially non-extensible. A typical non-extensible outer cover 49 can be manufactured from a thin plastic film or other flexible liquid-impermeable material. For example, the outer cover 49 may be formed from a polyethylene film having a thickness of from about 0.013 millimeter (0.5 mil) to about 0.051 millimeter (2.0 mils). If it is desired to present the outer cover 49 with a more clothlike feeling, the outer cover may comprise a polyolefin film having a nonwoven web laminated to the exterior surface thereof, such as a spunbond web of polyolefin fibers. For example, a stretch-thinned polypropylene film having a thickness of about 0.015 millimeter (0.6 mil) may have thermally laminated thereto a spunbond web of polypropylene fibers. The polypropylene fibers have a thickness of about 1.5 to 2.5 denier per filament, which nonwoven web has a basis weight of about 17 grams per square meter (0.5 ounce per square yard). The outer cover 49 may otherwise include bicomponent fibers such as polyethylene/polypropylene bicomponent fibers. Methods of forming such clothlike outer covers are known to those skilled in the art.

Further, the non-extensible outer cover 49 may be formed of a woven or nonwoven fibrous web layer which has been totally or partially constructed or treated to impart a desired level of liquid impermeability to selected regions that are adjacent or proximate the absorbent body 53. The non-extensible outer cover 49 may optionally be composed of a micro-porous “breathable” material which permits vapors to escape from the diaper 21 while still preventing liquid exudates from passing through the outer cover. For example, the non-extensible outer cover 49 may include a vapor permeable non-woven facing layer laminated to a micro-porous film. Suitable “breathable” outer cover 49 materials are described in U.S. Pat. No. 5,695,868 issued to McCormack et al. and U.S. Pat. No. 5,843,056 issued Dec. 1, 1998 to Good et al., the descriptions of which are hereby incorporated by reference. The non-extensible outer cover 49 can also be embossed or otherwise provided with a matte finish to provide a more aesthetically pleasing appearance.

The bodyside liner 51 is preferably pliable, soft feeling, and nonirritating to the wearer's skin, and is employed to help isolate the wearer's skin from the absorbent body 53. The liner 51 is less hydrophilic than the absorbent body 53 to present a relatively dry surface to the wearer, and is sufficiently porous to be liquid permeable to thereby permit liquid to readily penetrate through its thickness. A suitable bodyside liner 51 may be manufactured from a wide selection of web materials, but is preferably capable of stretching in at least one direction (e.g., longitudinal or lateral). Various woven and nonwoven fabrics including either or both synthetic and natural fibers can be used for the liner 51. For example, the bodyside liner 51 may be composed of a meltblown or spunbonded web of the desired fibers, and may also be a bonded-carded-web. Layers of different materials that may have different fiber deniers can also be used. The various fabrics can be composed of natural fibers, synthetic fibers or combinations thereof.

The bodyside liner 51 may be composed of a substantially hydrophobic material, and the hydrophobic material may optionally be treated with a surfactant or otherwise processed to impart a desired level of wettability and hydrophilicity. Examples of suitable materials for the bodyside liner 51 include 0.3-0.5 osy (10-17 gsm) polypropylene spun bond web treated with a suitable wettability treatment, 0.3-0.5 osy (10-17 gsm) bonded carded web and 0.4-0.8 osy (14-27 gsm) thru air bonded carded web. The fabric can be surface treated with an operative amount of surfactant, such as about 0.3 weight percent of a surfactant commercially available from Hodgson Textile Chemicals, Inc. under the trade designation AHCOVEL Base N-62. The surfactant can be applied by any conventional means, such as spraying, printing, brush coating or the like.

In particular embodiments, the top liner 51 is desirably extensible and capable of extending along with the outer cover 49 for desired fit of the diaper on the wearer. For example, the top liner 51 can be composed of various extensible materials such as a necked fabric, a creped fabric, a micro-pleated fabric, perforated polymer films or the like, as well as combinations thereof. The fabrics may be woven or nonwoven materials, such as spunbond fabrics, that may be elastic or non-elastic. Examples of suitable manufacturing techniques and suitable necked nonwoven fabric materials for such an extensible top liner 51 are described in U.S. Pat. No. 4,965,122 entitled REVERSIBLY NECKED MATERIAL, by M. T. Morman which issued Oct. 23, 1990.

Desirably, the bodyside liner 51 is made from non-elastic neckable materials for reduced cost and improved manufacturing efficiency. Suitable non-elastic neckable materials for such a configuration include nonwoven webs, woven materials and knitted materials. Such webs can include one or more fabric layers. Nonwoven fabrics or webs have been formed from many processes, for example, bonded carded web processes, meltblowing processes and spunbonding processes. The non-elastic neckable material is preferably formed from at least one member selected from fibers and filaments of inelastic polymers. Such polymers include polyesters, for example, polyethylene terephthalate, polyolefins, for example, polyethylene and polypropylene, polyamides. These fibers or filaments are used alone or in a mixture of two or more thereof. Suitable fibers for forming the neckable material include natural and synthetic fibers as well as bicomponent, multi-component, and shaped polymer fibers. Many polyolefins are available for fiber production according to the present invention, for example, fiber forming polypropylenes include Exxon Chemical Company's Escorene PD 3445 polypropylene and Himont Chemical Company's PF-304. Polyethylenes such as Dow Chemical's ASPUN 6811A linear low density polyethylene, 2553 LLDPE and 25355 and 12350 high density polyethylene are also suitable polymers.

The neckable material may be necked to form the extensible bodyside liner 51 by conventional necking processes which typically vary the surface speed of the web to draw or neck the material. Such necking will allow the material to extend in the transverse direction. As discussed above, such necked non-woven fabric materials typically are capable of being necked up to about 80 percent. For example, the liner 51 may be necked from about 10 to about 80 percent, more desirably from about 20 to about 60 percent, and still more desirably from about 30 to about 50 percent for improved performance.

The absorbent body 53 may have any of a number of shapes, including rectangular, I-shaped, or T-shaped and is desirably narrower in the crotch region than in the front or back regions of the diaper 21. As illustrated herein, the absorbent body 53 is generally T-shaped with the laterally extending crossbar of the “T” generally corresponding to the front region 25 of the diaper 21 for improved performance, especially for male infants. The size and the absorbent capacity of the absorbent body 53 will be selected according to the size of the intended wearer and the liquid loading imparted by the intended user of the diaper. In addition, it has been found that the densities and/or basis weights of the absorbent body 53 can be varied. In the embodiment described herein, the absorbent body 53 has an absorbent capacity of at least about 100 grams of synthetic urine.

The absorbent body 53 of the illustrated embodiment includes three layers, however it is to be understood that the absorbent body may have one, two or more than three layers without departing from the scope of the present invention. A central absorbent layer 63 of the absorbent body 53 preferably includes hydrophilic fibers and superabsorbent particles, as described more fully below. A ventilation layer 65 is disposed between the central absorbent layer 63 and the outer cover 49 to insulate the outer cover 49 from the absorbent body 53, to improve air circulation and to effectively reduce the dampness of the garment facing surface of the outer cover. The ventilation layer 65 also assists in distributing fluid exudates to portions of the absorbent body 53 that do not directly receive an insult. A surge management layer 67 is disposed between the bodyside liner 51 and the central absorbent layer 63 to prevent pooling of the liquid exudates and to further improve air exchange and distribution of the liquid exudates within the diaper 21.

Various types of wettable, hydrophilic fibrous material can be used to form part of the central absorbent layer 63. Examples of suitable fibers include naturally occurring organic fibers composed of intrinsically wettable material, such as cellulosic fibers; synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers; and synthetic fibers composed of a nonwettable thermoplastic polymer, such as polypropylene fibers, which have been hydrophilized by appropriate means. The fibers may be hydrophilized, for example, by treatment with silica, treatment with a material which has a suitable hydrophilic moiety and is not readily removable from the fiber, or by sheathing the nonwettable, hydrophobic fiber with a hydrophilic polymer during or after the formation of the fiber. For the purposes of the present invention, it is contemplated that selected blends of the various types of fibers mentioned above may also be employed.

The central absorbent layer 63 of the absorbent body 53 may include a combination of hydrophilic fibers and high-absorbency material. However, it is understood that absorbent bodies having absorbent layers of other compositions and having dimensions other than described may be used without departing from the scope of the present invention. More specifically, the high-absorbency material in the central absorbent layer 63 can be selected from natural, synthetic, and modified natural polymers and materials. The high-absorbency materials can be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the central absorbent layer 63 of the absorbent body 53 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and the like. Mixtures of natural and wholly or partially synthetic absorbent polymers can also be useful in the present invention.

The high absorbency material may be in any of a wide variety of geometric forms. As a general rule, it is preferred that the high absorbency material be in the form of discrete particles or beads. However, the high absorbency material may also be in the form of fibers, flakes, rods, spheres, needles, or the like. In general, the high absorbency material is present in the central layer 63 of the absorbent body 53 in an amount of from about 5 to about 90 percent by weight, desirably in an amount of at least about 30 percent by weight, and even more desirably in an amount of at least about 50 percent by weight based on a total weight of the central layer 31. For example, the central absorbent layer 63 may include a laminate which includes at least about 50 percent by weight and desirably at least about 70 percent by weight of high-absorbency material overwrapped by a fibrous web (not shown) or other suitable material for maintaining the high-absorbency material in a localized area.

An example of high-absorbency material suitable for use in the central absorbent layer 63 is SANWET IM 3900 polymer available from Hoechst Celanese, a business having offices in Portsmouth, Va. Other suitable superabsorbents may include FAVOR SXM 880 polymer obtained from Stockhausen, a business having offices in Greensboro, N.C. Optionally, a substantially hydrophilic tissue wrapsheet (not shown) may be employed to help maintain the integrity of the structure of the central absorbent layer 63. The tissue wrapsheet is typically placed about the layer 63 over at least the two major facing surfaces thereof. The tissue wrapsheet can be made of an absorbent cellulosic material, such as creped wadding or a high wet-strength tissue. The tissue wrapsheet can be configured to provide a wicking layer that helps to rapidly distribute liquid over the mass of absorbent fibers constituting the central absorbent layer 63.

The surge management layer 67 is typically less hydrophilic than the central absorbent layer 63, and has an operable level of density and basis weight to quickly collect and temporarily hold liquid surges, to transport the liquid from its initial entrance point and to substantially completely release the liquid to other parts of the central absorbent layer. This configuration can help prevent the liquid from pooling and collecting on the portion of the diaper 21 positioned against the wearer's skin, thereby reducing the feeling of wetness by the wearer. The structure of the surge management layer 67 also generally enhances the air exchange within the diaper 21.

Various woven and nonwoven fabrics can be used to construct the surge management layer 67. For example, the surge management layer 67 may be a layer made of a meltblown or spunbond web of synthetic fibers, such as polyolefin fibers. The surge management layer 67 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge management layer 67 may be composed of a substantially hydrophobic material, and the hydrophobic material may optionally be treated with a surfactant or otherwise processed to impart a desired level of wettability and hydrophilicity. As one example, the surge management layer 67 includes a hydrophobic, nonwoven material having a basis weight of from about 30 to about 120 grams per square meter.

The central absorbent layer 63 of the absorbent body 53 is positioned in liquid communication with surge management layer 67 to receive liquids released from the surge management layer, and to hold and store the liquid. In the illustrated embodiment, the surge management layer 67 is a separate layer positioned over the central absorbent layer 63 of the absorbent body 53. The surge management layer 67 serves to quickly collect and temporarily hold discharged liquids, to transport such liquids from the point of initial contact and spread the liquid to other parts of the surge management layer 67, and then to substantially completely release such liquids into the central absorbent layer 63 of the absorbent body 53.

The surge management layer 67 can be of any desired shape. Suitable shapes include for example, circular, rectangular, triangular, trapezoidal, oblong, dog-boned, hourglass-shaped, or oval. The surge management layer 67 of the illustrated embodiment is coextensive with the central absorbent layer 63 of the absorbent body 53. Alternatively, the surge management layer 67 may extend over only a part of the central absorbent layer 63. Where the surge management layer 67 extends only partially along the length of the central absorbent layer 63, the surge management layer may be selectively positioned anywhere along the central layer. For example, the surge management layer 67 may function more efficiently when it is offset toward the front 25 of the diaper 21. The surge management layer 67 may also be approximately centered about the longitudinal center line of the central absorbent layer 63 of the absorbent body 53.

Additional materials suitable for the surge management layer 67 are set forth in U.S. Pat. No. 5,486,166 issued Jan. 23, 1996 in the name of C. Ellis et al. and entitled “FIBROUS NONWOVEN WEB SURGE LAYER FOR PERSONAL CARE ABSORBENT ARTICLES AND THE LIKE”; U.S. Pat. No. 5,490,846 issued Feb. 13, 1996 in the name of Ellis et al. and entitled “IMPROVED SURGE MANAGEMENT FIBROUS NONWOVEN WEB FOR PERSONAL CARE ABSORBENT ARTICLES AND THE LIKE”; and U.S. Pat. No. 5,364,382 issued Nov. 15, 1994 in the name of Latimer et al. and entitled “ABSORBENT STRUCTURE HAVING IMPROVED FLUID SURGE MANAGEMENT AND PRODUCT INCORPORATING SAME”, the disclosures of which are hereby incorporated by reference.

The ventilation layer 65 may be formed from materials described above as being suitable for the surge management layer 67 such as nonwoven, (e.g., spunbond, meltblown or carded), woven, or knitted fibrous webs composed of natural fibers and/or synthetic polymeric fibers. Suitable fibers include, for example, acrylic fibers, polyolefin fibers, polyester fibers, or blends thereof. The ventilation layer 65 may also be formed from a porous foam material such as an open-celled polyolefin foam, a reticulated polyurethane foam, and the like. The ventilation layer 65 may include a single layer of material (as shown) or a composite of two or more layers of material. As one example, the ventilation layer 65 includes a hydrophobic, nonwoven material having a thickness of at least about 0.10 centimeters determined under a restraining pressure of 0.05 psi (0.34 kPa) and a basis weight of from about 20 to about 120 grams per square meter. However, the ventilation layer 65 may comprise a bonded-carded-web, nonwoven fabric that includes bicomponent fibers and that has an overall basis weight of about 83 grams per square meter. The ventilation layer 65 can be a homogeneous blend of about 60 weight percent polyethylene/polyester (PE/PET), sheath-core bicomponent fibers that have a fiber denier of about 3 d and about 40 weight percent single component polyester fibers that have a fiber denier of about 6 d and that have fiber lengths of from about 3.8 to about 5.08 centimeters.

The ventilation layer 65 can be of any desired shape. Suitable shapes include, for example, circular, rectangular, triangular, trapezoidal, oblong, dog-boned, hourglass-shaped, or oval. The ventilation layer 65 may extend beyond, completely over or partially over the central absorbent layer 63 of the absorbent body 53. For example, the ventilation layer 65 may suitably be located over the crotch region 27 of the diaper 21 and be substantially centered side-to-side with respect to the lateral axis Y of the diaper 21. It is generally desired that the entire absorbent body 53 be underlaid with the ventilation layer 65 to prevent substantially all surface to surface contact between the outer cover 49 and the central absorbent layer 63. In the illustrated embodiment, the ventilation layer 65 is coextensive with the central absorbent layer 63. This allows for the maximum degree of air exchange with minimal dampness on the garment facing (e.g., outer) surface of the outer cover 49.

The ventilation layer 65 is arranged in a direct, contacting liquid communication with the central absorbent layer 63 of the absorbent body 53. The ventilation layer 65 may be operably connected to the outer cover 49 with a conventional pattern of adhesive (not shown), such as a swirl adhesive pattern. In addition, the ventilation layer 65 may be operably connected to the central absorbent layer 63 with a conventional pattern of adhesive. The amount of adhesive add-on should be sufficient to provide the desired levels of bonding, but should be low enough to avoid excessively restricting the movement of air and vapor from the central absorbent layer 63 and through the outer cover 49.

The ventilation layer 65 may further serve to quickly collect and temporarily hold discharged liquids, which pass through the central absorbent layer 63 of the absorbent body 53. The ventilation layer 65 may then transport such liquids from the point of initial contact and spread the liquid to other parts of the ventilation layer, and then substantially completely release such liquids back into the central absorbent layer 63. Thus, in the illustrated embodiment, the absorbent body 53 includes three layers. It is to be understood that although preferred, the ventilation layer 65 and surge management layer 67 may be omitted from the absorbent body 53 without departing from the scope of the present invention.

Fastener tabs 41 (FIGS. 1 and 3) are secured to the central absorbent assembly 23 generally at the back region thereof with the tabs extending laterally outward from the opposite side edges of the assembly. The fastener tabs 41 may be attached to the outer cover 49, to the bodyside liner 51, between the outer cover and liner, or to other components of the diaper 21. The tabs 41 may also be elastic or otherwise rendered elastomeric. For example, the fastener tabs 41 may be an elastomeric material such as a neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. Examples of articles that include selectively configured fastener tabs are described in U.S. Pat. No. 5,496,298 issued Mar. 5, 1996 to Kuepper et al.; U.S. Pat. No. 5,540,796 to Fries; and U.S. Pat. No. 5,595,618 to Fries; the disclosures of which are also incorporated herein by reference. Alternatively, the fastener tabs 41 may be formed integrally with a selected diaper component. For example, the tabs 41 may be formed integrally with the inner or outer layer 57, 55 of the outer cover 49, or with the bodyside liner 51.

Fastening components, such as hook and loop fasteners, designated 71 and 72 respectively, are employed to secure the diaper 21 on the body of a child or other wearer. Alternatively, other fastening components (not shown), such as buttons, pins, snaps, adhesive tape fasteners, cohesives, mushroom-and-loop fasteners, or the like, may be employed. Desirably, the interconnection of the fastening components 71, 72 is selectively releasable and re-attachable. In the illustrated embodiment, the hook fasteners 71 are secured to and extend laterally out from the respective fastener tabs 41 at the back region 29 of the diaper 21. However, it is understood that the fastener tabs 41 may be formed of a hook material and thus comprise the hook fasteners 71 without departing from the scope of this invention.

The loop fastener 72 of the illustrated embodiment is a panel of loop material secured to the outer cover 49 at the front region 25 of the diaper 21 to provide a “fasten anywhere” mechanical fastening system for improved fastening of the hook fasteners 71 with the loop fastener. The loop material may include a pattern-unbonded nonwoven fabric having continuous bonded areas that define a plurality of discrete unbonded areas. The fibers or filaments within the discrete unbonded areas of the fabric are dimensionally stabilized by the continuous bonded areas that encircle or surround each unbonded area, such that no support or backing layer of film or adhesive is required. The unbonded areas are specifically designed to afford spaces between fibers or filaments within the unbonded areas that remain sufficiently open or large to receive and engage hook elements of the complementary hook fasteners 71. In particular, a pattern-unbonded nonwoven fabric or web may include a spunbond nonwoven web formed of single component or multi-component melt-spun filaments. For example, the loop material may be a laminated structure including a polyethylene component and a polypropylene component adhesively bonded together with the polypropylene component facing outward away from the outer cover 49 to receive the hook fasteners 71. Examples of suitable pattern-unbonded fabrics are described in U.S. Pat. No. 5,858,515 issued Jan. 12, 1999 to T. J. Stokes et al. and entitled PATTERN-UNBONDED NONWOVEN WEB AND PROCESS FOR MAKING THE SAME; the entire disclosure of which is incorporated herein by reference in a manner that is consistent herewith.

The diaper 21 shown in FIG. 1 also comprises a pair of containment flaps, generally indicated at 75, configured to provide a barrier to the lateral flow of body exudates. The containment flaps 75 are located generally adjacent the laterally opposite side edges 31 of the diaper 21 and, when the diaper is laid flat as shown in FIGS. 1 and 2, extend inward toward the longitudinal axis X of the diaper. Each containment flap 75 typically has a free, or unattached end 77 free from connection with the bodyside liner 51 and other components of the diaper 21. Elastic strands 79 disposed within the flaps 75 adjacent the unattached ends thereof urge the flaps toward an upright, perpendicular configuration in at least the crotch region 27 of the diaper 21 to form a seal against the wearer's body when the diaper is worn. The containment flaps 75 may extend longitudinally the entire length of the absorbent body 53 or they may extend only partially along the length of the absorbent body. When the containment flaps 75 are shorter in length than the absorbent body 53, the flaps can be selectively positioned anywhere between the side edges 31 of the diaper 21 in the crotch region 27. In a particular aspect of the invention, the containment flaps 75 extend the entire length of the absorbent body 53 to better contain the body exudates.

Such containment flaps 75 are generally well known to those skilled in the art and therefore will not be further described herein except to the extent necessary to describe the present invention. As an example, suitable constructions and arrangements for containment flaps 75 are described in U.S. Pat. No. 4,704,96 issued Nov. 3, 1987, to K. Enloe, the disclosure of which is hereby incorporated by reference. The diaper 21 may also incorporate other containment components in addition to or instead of containment flaps 75. For example, while not shown in the drawings, other suitable containment components may include, but are not limited to, elasticized waist flaps, foam dams in the front, back and/or crotch regions, and the like.

The various components of the diaper 21 are integrally assembled together using a suitable form of attachment, such as adhesive, sonic bonds, thermal bonds or combinations thereof. In the illustrated embodiment, the outer cover 49 and absorbent body 53 are attached to each other with lines of adhesive 81, such as a hot melt or pressure-sensitive adhesive. The bodyside liner 51 is also connected to the outer cover 49 and may also be connected to the absorbent body 53 using the same forms of attachment. However, the bodyside liner 51 is desirably free of fixed connected to the outer cover 49 and absorbent body 53 in the crotch region 27 of the diaper 21. The bodyside liner 51 may be connected to the outer cover 49 at the lateral edge margins of the crotch region 27, but at least the central portion is free of such connection. Rather than being entirely free of such connection, the bodyside liner 51 may be connected to the absorbent body 53 in the crotch region 27 by a light adhesive 83 which will break away in use. Preferably, connection of the bodyside liner 51 to the outer cover 49 is limited to overlying peripheral edge margins of the two to promote independent stretching movement of the liner and cover relative to each other. If the diaper 21 is to be sold in a prefastened condition, the diaper may also have passive bonds (not shown) which join the back region 29 with the front region 25.

Examples of other diaper configurations suitable for use in connection with the instant application that may or may not include diaper components similar to those described previously are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,176,672 issued Jan. 5, 1993, to Bruemmer et al.; U.S. Pat. No. 5,192,606 issued Mar. 9, 1993, to Proxmire et al., and U.S. Pat. No. 5,509,915 issued Apr. 23, 1996 to Hanson et al., the disclosures of which are herein incorporated by reference.

To provide improved fit and to help further reduce leakage of body exudates from the diaper 21, elastic components are incorporated into the diaper, particularly at the waist and leg openings 43, 37 thereof. For example, the diaper 21 shown in FIG. 1 has waist elastic components, generally indicated at 99 in FIGS. 1 and 3, disposed generally adjacent the ends or waist edges 33, 35 of the diaper to provide elasticized waistbands. The waist elastic components 99 are configured to laterally gather and shirr the end margins of the diaper 21 to provide a resilient, comfortably close fit around the waist of the wearer.

As shown in FIGS. 1 and 2, leg elastic components, generally indicated at 101, are secured between the bodyside liner 51 and the inner layer 55 of the outer cover 49, such as by adhesive 81 generally at the edge margins of the laterally opposite side edges 31 of the diaper 21. It is understood however that the leg elastic components 101 may instead be secured between the outer and inner layers 55, 57 of the outer cover 49, such as by adhesive 59, without departing from the scope of this invention.

Each leg elastic component 101 extends continuously generally adjacent a respective side edge 31 of the diaper 21 from the front region 25 through the crotch region 27 to the back region 29 of the diaper 21. More particularly, each leg elastic component 101 extends continuously generally along a path having an outer boundary 103 and an inner boundary 105 which together define a width W of the elastic component as the component extends longitudinally along the path. As shown in FIG. 1, the width W of the elastic component 101 is substantially greater at the back region 29 of the diaper 21 than at the crotch region 27 and at least a portion of the front region 29 of the diaper. For example, the width W of the elastic component 101 of FIG. 1 is approximately 1.25 inches at the back region 29 of the diaper and narrows to about 0.625 inches at the crotch region 27. As a result, upon longitudinal elongation (e.g., stretching) of the elastic component 101, such as when the diaper 21 is laid flat as shown in FIG. 1 or otherwise worn by a wearer, the elastic component has a non-uniform elastic tension, and more particularly an elastic tension at the back region 29 of the diaper is substantially greater than an elastic tension at the crotch region 27 and front region 25 of the diaper. It is also contemplated that the elastic tension of the elastic component 101 may be greater at the front region 25 of the diaper than the elastic tension at the back region 29 and/or crotch region 27 of the diaper without departing from the scope of this invention.

With particular reference still to FIGS. 1 and 2, each elastic component 101 generally comprises an elongate substrate 107, such as a sheet or ribbon, having threads or strands 109a, 109b of elastic material secured to the substrate in generally parallel, spaced relationship with each other. As an example, one suitable elastic material from which the elastic strands 109a, 109b may be constructed is a dry-spun coalesced multi-filament elastomeric thread sold under the trade name LYCRA® and available from E. I. du Pont de Nemours and Company, Wilmington, Del., U.S.A. The elastic strands 109a, 109b are desirably secured to the substrate 107 while in a stretched (e.g., elastically contractible) condition such that the retractive forces of the elastic strands tend to gather the substrate. The substrate 107 is in turn secured between the outer and inner layers 55, 57 of the outer cover 49 with the substrate ungathered (e.g., with the elastic strands 109a, 109b in a stretched condition) such that the retractive forces of the elastic strands gather the diaper 21 at the leg openings 37 to provide a snug fit around the wearer's leg.

Each elastic component 101 shown in FIG. 1 comprises five elastic strands 109a, 109b extending in parallel, spaced relationship with each other on the substrate. The elastic strands 109a, 109b are desirably spaced apart at least about 0.125 inches and more particularly at least about 0.25 inches. For purposes of comparison, for conventional diapers that incorporate multiple elastic strands as leg elastics, the elastic strands are typically spaced less than or equal to about 0.10 inches apart. The innermost and outermost elastic strands 109a, 109b of the elastic component 101 are also spaced slightly inward from the sides of the substrate 107 such that the inner and outer boundaries 103, 105 of the elastic component are respectively defined at least in part by the sides of the substrate. For example, the innermost elastic strand 109a is spaced in the range of about 0 to about 0.25 inches inward from the inner side of the substrate 107 and the outermost elastic strand 109b is spaced in the range of about 0 to about 0.25 inches from the outer side of the substrate.

In the illustrated embodiment, the innermost three strands 109a of the elastic component 101 extend continuously along the length of the path of the elastic component from the front region 25 through the crotch region 27 to the back region 29 of the diaper 21 while the outermost two strands 109b extend only partially along the length of the path. More particularly, the outermost two strands 109b extend only within the back region 29 and a portion of the front region 25 of the diaper 21, and are otherwise generally omitted from (e.g., are discontinuous in or do not extend within) the crotch region 27 and a portion of the front region of the diaper. Thus, as described previously, the elastic component 101 has a greater width W, and therefore a greater elastic tension, in the back region 29 of the diaper than in the crotch region 27 and at least a portion of the front region 25 of the diaper.

It is understood that where the elastic component 101 comprises elastic strands 109a, 109b or threads, such as in the illustrated embodiment of FIG. 1, the elastic component may comprise more or less than five elastic strands secured to the substrate 107, but desirably has at least two such strands, without departing from the scope of this invention. For example, at least one strand 109a extends continuously along the path of the elastic component 101 from the front region 25 through the crotch region 27 to the back region 29 of the diaper 21 while at least one other strand 109b is discontinuous or otherwise extends only partially along the length of the path. It is also contemplated that the elastic components 101 may comprise elastic strands 109a, 109b secured directly to existing components within the diaper 21, e.g., without being first secured to a separately constructed sheet, ribbon or other suitable substrate 107. In such an arrangement, the inner and outer boundaries 105, 103 of the path of the elastic components 101 are respectively defined by the innermost and outermost strands 109a, 109b extending along the path.

It is further contemplated that the elastic component 101 may be constructed other than of individual elastic strands 109a, 109b. For example, elastic sheets or ribbons (not shown) made from natural rubber, synthetic rubber or thermoplastic elastomeric polymers may be used without departing from the scope of this invention. In such a configuration, the inner and outer boundaries 105, 103 of the elastic components 101 are respectively defined by the laterally opposite sides of the elastic sheet or ribbon. Portions of the elastic sheet or ribbon may also be removed, such as along the segment thereof which extends through the crotch region 27 of the diaper 21, to vary the width W of the elastic component as well as the overall width of the diaper along the length of the path of the elastic component to thereby create an elastic tension gradient along the path of the elastic component upon elongation thereof.

The path of the elastic component 101 of the illustrated embodiment is generally non-parallel relative to the longitudinal axis X of the diaper 21, and more particularly the path curves laterally inward as its transitions from the back region 29 into the crotch region 27 of the diaper and then curves laterally outward as the elastic component transitions from the crotch region to the front region 25 of the diaper. Thus, as shown in FIG. 1, each of the inner and outer boundaries 105, 103 of the elastic component 101 has a maximum laterally outward position (e.g., as measured from the longitudinal axis X, or centerline, of the diaper 21) located generally at the back region 29 of the diaper and a minimum laterally outward position located generally at the crotch region 27 of the diaper. An applied, or inner curvature 111 of the elastic component 101 is defined herein as the difference between the maximum laterally outward position and the minimum laterally outward position of the inner boundary 105 of the elastic composite 101. An actual, or outer curvature 113 of the elastic composite 101 is defined herein as the difference between the maximum laterally outward position and the mininum laterally outward position of the outer boundary 103 of the elastic component as measured from the longitudinal axis X of the diaper 21.

The actual, or outer curvature 113 of the elastic component 101 shown in FIG. 1 is greater than the applied, or inner curvature 111 of the elastic component. For example, the outer curvature 113 of the leg elastic component 101 of the illustrated embodiment is about 1.25 inches and the inner curvature 111 is about 0.625 inches. For comparison purposes, conventional diapers that incorporate leg elastics typically have an outer curvature of less than or equal to about one inch. It is understood that the outer curvature 113 of the leg elastic component 101 may be less than or greater than 1.25 inches but is desirably greater than one inch. It is further contemplated that the inner curvature 111 of the elastic component 101 may instead be equal to the outer curvature 113 of the elastic component, or it may be substantially zero (e.g., the inner boundary 105 extends generally parallel to the longitudinal axis X of the diaper 21) without departing from the scope of this invention.

In accordance with one method for making an absorbent article such as the diaper 21 shown in FIG. 1, the outer cover 49 and bodyside liner 51 are initially configured to be generally rectangular in shape (e.g., without contoured leg openings 37). The various components of the diaper 21 are assembled in a generally conventional manner with the leg elastic components 101 being secured within the diaper, e.g., between the bodyside liner 51 and the inner layer 55 of the outer cover 49, along the desired path. For example, the leg elastic components 101 are desirably secured within the diaper 21 in accordance with the applied, or inner curvature 111 of the leg elastic components. The leg elastic components 101 are desirably initially of a generally uniform width, e.g., in the illustrated embodiment all five elastic strands 109a, 109b would extend continuously along the path of each leg elastic component. The leg openings 37 are then cut into the laterally opposite side edges 31 of the diaper 21 to form the desired shape of the leg openings. Desirably, a segment of each leg elastic component 101 is cut concurrently with the side edges 31 of the diaper 21 so as to generally narrow the width W of the elastic component 101 along that segment and to define the desired actual, or outer curvature 113 of the leg elastic component. More desirably, a segment of each leg elastic component 101 which extends through the crotch region 27 and at least a portion of the front region 29 of the diaper 21 is cut to reduce the width W of the elastic component within those regions.

For example, the diaper shown in FIG. 1 is initially constructed to have five elastic strands 109a, 109b extending continuously from the front region 25 through the crotch region 27 to the back region 29 of the diaper 21. When the laterally opposite side edges 31 of the diaper 21 are cut to form the leg openings 37, the two outermost strands 109b are also cut, such as in the crotch region 27 and a portion of the front region 29 of the diaper, to reduce the width W of the path of the elastic component 101 (e.g., from five strands down to three strands) within those regions and to define the outer curvature 113 of the elastic component. Such an arrangement provides the desired elastic tension gradient within the elastic component 101 as it extends along the path.

Forming the diaper 21 in this manner also positions the outer boundary 103 of each leg elastic component 101 close to the respective side edge 31 of the diaper 21 to reduce the amount of ungathered material at the side edges. As an example, the outer boundary 103 of each leg elastic component 101 is desirably spaced about zero inches (as shown in FIG. 1, which otherwise means that the outer boundary is co-linear with the side edge 31 of the diaper) to about 0.375 inches inward from the respective side edge of the diaper, and more preferably about zero inches to about 0.25 inches. For comparison purposes, the leg elastics of conventional diapers are spaced inward from the side edges 31 of the diaper a distance of about 0.75 inches or more.

### EXAMPLE

A Huggies® Ultratrim diaper, which is available from Kimberly-Clark of Neenah, Wis., U.S.A, was tested to measure the elastic tension in the leg elastics of the diaper at 90% elongation thereof. The tested diaper incorporated four elastic strands extending continuously in parallel spaced relationship with each other from the front region 25 through the crotch region 27 to the back region 29 adjacent the laterally opposite side edges 31 of the diaper 21. Another diaper 21, constructed in accordance with the present invention to have a tension gradient along the path of the elastic component 101, and more particularly in accordance with the diaper shown in FIG. 1, was also tested to measure the elastic tension in the leg elastic components of the diaper at 90% elongation thereof.

The elastic tension test used to measure the elastic tension at 90% elongation was as follows. Each diaper 21 was first hung from a conventional lightbox in an unfolded, vertical orientation with one end (e.g., the back end 35) of the diaper up and the outer cover 49 of the diaper facing laterally outward away from the lightbox. The lightbox was configured with a pair of fixed upper clamps spaced approximately 5.5 inches apart for holding the end of the diaper generally at the waistband. The waistband at the back end 35 of the diaper 21 was generally fully stretched (e.g., to eliminate gathers in or otherwise straighten the waistband) and the clamps were clamped to the waistband without clamping any of the absorbent body 35 therein.

The waistband at the opposite end e.g., the front end 33, of the diaper 21 was then generally fully stretched (i.e., to eliminate gathers in or otherwise straighten the waistband) and a clamp weight was secured to the waistband at the front end of the diaper so that the diaper hung freely from the upper clamps in a longitudinally elongated configuration. The clamp weight was approximately 1,000 grams total mass and included a pair of clamps spaced approximately 5.5 inches apart at their centerlines (e.g., spaced approximately the same as the clamps attached to the back end of the diaper) for attaching the clamp weight to the front end of the diaper.

With the diaper 21 in this longitudinally elongated configuration, a marking was made on each of the left and right and sides of the diaper to delineate the longitudinal center of the diaper. A pair of markings was then made on the left hand side of the diaper above the corresponding longitudinal centerline marking (e.g., in the back half of the diaper). The first of these markings was spaced longitudinally from the diaper centerline approximately 15 mm and the second of these markings was spaced longitudinally approximately 80 mm beyond (e.g. nearer the back end of the diaper) the first marking. In this manner, the portion of the left side leg elastics extending longitudinally between the pair of markings extended within both the crotch region 27 and the back region 29 of the diaper. A similar pair of markings was made on the left side of the diaper below the diaper centerline (e.g., within the front half of the diaper). That is, a first marking was spaced longitudinally below the diaper centerline approximately 15 mm and a second marking was spaced longitudinally from the first marking approximately 80 mm. In this manner, the portion of the left side leg elastics extending longitudinally between the pair of markings extended within both the crotch region 27 and the front region 25 of the diaper. Two pair of markings were similarly made on the right hand side of the diaper.

The clamp weight was then removed from the diaper 21 and the diaper was removed from the lightbox. A first elastic specimen was cut from the diaper 21, e.g., from the upper (i.e., back) left side of the diaper, by first cutting laterally in from the side edge of the diaper at approximately 13 mm longitudinally beyond one of the markings (e.g., toward the back end of the diaper), to a lateral position inward of the elastic strands. A subsequent cut was made lengthwise from the first cut to approximately 13 mm beyond the other marking (e.g., nearer the diaper centerline) and then a final cut was made laterally in from the side edge of the diaper to free the specimen from the diaper 21. A second elastic specimen was cut from the lower (i.e., front) left side of the diaper in substantially the same manner. A pair of elastic specimens were also cut from the right side of the diaper in a like manner.

Each elastic specimen was then secured in a testing device by a generally fixed upper clip and a generally moveable lower clip, both of which are constructed to inhibit the specimen against slipping or becoming damaged upon tensioning the specimen. More particularly, the specimen was first secured at one end by the upper clip, with the specimen marking near the one end being aligned with the leading edge (e.g., the lowermost edge) of the upper clip so that the specimen hung freely from the upper clip in a generally relaxed (e.g., unstretched) condition. The weight of the specimen was tared and then the other end of the specimen was secured by the lower clip, with the marking near this end of the specimen being aligned with the leading edge (e.g., uppermost edge) of the lower clip. The lower clip was moved longitudinally away from the fixed upper clip until the specimen was elongated longitudinally to about 90% of the previously achieved longitudinally elongated configuration (e.g., to a configuration in which the spacing between the markings on the specimen was about 72 mm, or 90% of 80 mm. The specimen was maintained in this condition for about thirty seconds. The elastic tension was then measured and recorded using a suitable force gauge.

For the Huggies® diaper, the average elastic tension in the leg elastics was measured to be about 76.7 grams at the back region 29 of the diaper and about 77.5 grams at the front region 25 of the diaper. In contrast, for the diaper 21 constructed in accordance with the present invention, the average elastic tension in the leg elastic component 101 was measured to be about 117.8 grams at the back region 29 of the diaper and about 90.5 grams at the front region 25 of the diaper. Thus, the elastic tension is substantially uniform at the front and back regions 25, 29 of the Huggies® diaper whereas the elastic tension at the back region of the diaper 21 constructed in accordance with the present invention is approximately 30% greater than the elastic tension at the front region of the diaper.

Providing an elastic tension gradient along the continuous path of each leg elastic component 101, and more particularly providing an elastic tension which is greater at the back region 29 than the crotch region 27 and front region 25 of the diaper 21, reduces the amount of red-marking on the skin of the wearer while maintaining sufficient retraction to provide a snug fit and to inhibit leakage. Increasing the spacing between the elastic strands 109a, 109b of each leg elastic component 101 as compared to conventional diapers also reduces red-marking by spreading the elastic tension over a wider area of the wearer's skin.

Increasing the actual, or outer curvature 113 of the leg elastic components 101 and reducing the amount of ungathered material laterally outward of the elastic components (e.g., by cutting into the leg elastic components), substantially reduces the overall width of the diaper 21 in the crotch region 27 to increase comfort to the wearer. For example, the width of the crotch region 27 of the diaper 21 of FIG. 1 is about six inches. As a comparison, conventional diapers typically have a crotch width of about eight inches. Positioning the outer boundary 103 of each leg elastic component close to the respective side edge 31 of the diaper 21 also reduces the risk that the side edges will tuck inward when securing the diaper on a baby.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. An absorbent article comprising:
an anterior region;
a posterior region;
a central region interconnecting the anterior and posterior regions and extending longitudinally therebetween; and
an elastic component extending generally continuously along a path from the anterior region of said article through the central region to the posterior region thereof, said elastic component having an elastic tension which is substantially non-uniform along the path defined by the elastic component.

2. An absorbent article as set forth in claim 1 wherein the elastic tension of the elastic component generally at the posterior region of said article is substantially greater than the elastic tension of the elastic component at the anterior region.

3. An absorbent article as set forth in claim 1 wherein the elastic tension of the elastic component generally at the posterior region of said article is substantially greater than the elastic tension of the elastic component at the central region.

4. An absorbent article as set forth in claim 1 wherein the elastic component comprises at least two elongate elastic strands together defining the path along which the elastic component extends continuously from the anterior region through the crotch region to the posterior region of said article, at least one of said elastic strands extending continuously along the length of the path and at least one other elastic strand extending less than the full length of the path such that the elastic tension of the elastic component is substantially non-uniform along the length of the path.

5. An absorbent article as set forth in claim 4 wherein said at least one other strand is generally discontinuous within the crotch region of the article.

6. An absorbent article as set forth in claim 4 wherein said at least one other strand extends only partially within the front region of the article.

7. An absorbent article as set forth in claim 4 wherein the elastic component further comprises an elongate substrate extending from the anterior region through the central region to the posterior region of said article, the at least two elastic strands being secured to the substrate.

8. An absorbent article as set forth in claim 1 wherein the path defined by the elastic component has a width, said width being substantially non-uniform along at least a segment of said path.

9. An absorbent article as set forth in claim 8 wherein the width of the path defined by the elastic component is greater at the back region of the article than at the crotch region of the article.

10. An absorbent article as set forth in claim 8 wherein the width of the path defined by the elastic component is greater at the back region of the article than at least a portion of the front region of the article.

11. An absorbent article as set forth in claim 4 wherein the at least two elastic members are spaced from each other a distance equal to or greater than about 0.125 inches.

12. An absorbent article as set forth in claim 11 wherein the at least two elastic members are spaced from each other a distance in the range of about 0.125 inches to about 0.25 inches.

13. An absorbent article as set forth in claim 4 wherein the elastic component comprises at least five elongate elastic members extending generally longitudinally within the posterior region of said article, the elastic component further comprising less than five elastic members extending generally longitudinally within at least a portion of the anterior region of said article.

14. An absorbent article as set forth in claim 4 wherein the elastic component comprises the elastic component comprises at least five elongate elastic members extending generally longitudinally within the posterior region of said article, the elastic component further comprising less than five elastic members extending generally longitudinally within the central region of said article.

15. An absorbent article as set forth in claim 1 wherein said absorbent article has longitudinal ends defining the length of the article and laterally opposite side edges defining the width of the article, the elastic component defining an inner boundary and an outer boundary of the path along which the elastic component extends, said elastic component extending along said path generally adjacent at least one of the side edges of said article in closely spaced relationship therewith.

16. An absorbent article as set forth in claim 15 wherein the outer boundary of the path defined by the elastic component is generally co-linear with the at least one side edge of said article.

17. An absorbent article as set forth in claim 1 further comprising an outer cover, a liner adapted for contiguous relationship with the wearer of said article, and an absorbent body disposed between the outer cover and the liner for absorbing liquid body exudates, longitudinal ends and laterally opposite side edges, the elastic component being a first elastic component and extending generally continuously along a path generally adjacent one of said side edges from the anterior region through the crotch region to the posterior region of said article, said article further comprising a second elastic component extending generally continuously along a path generally adjacent the opposite one of said side edges from the anterior region through the crotch region to the posterior region of said article, the second elastic component having an elastic tension which is substantially non-uniform along the path defined by said second elastic component.

18. An absorbent article as set forth in claim 17 wherein the absorbent article is a diaper wherein the laterally opposite side edges of the diaper define the width of said diaper, the diaper having a minimum width generally within the crotch region of less than or equal to about six inches.

19. An absorbent article having a longitudinal axis and comprising:
an anterior region;
a posterior region;
a central region interconnecting the anterior and posterior regions and extending longitudinally therebetween; and
an elastic component extending generally continuously along a path from the anterior region of said article through the central region to the posterior region thereof, said elastic component defining an outer boundary of said path, at least a portion of the outer boundary extending generally non-parallel to the longitudinal axis of said article and defining an outer curvature of the elastic component which is greater than one inch.

20. An absorbent article as set forth in claim 19 wherein the elastic component has an elastic tension which is substantially non-uniform along the length of the path defined by said elastic component.

21. An absorbent article as set forth in claim 20 wherein the elastic tension of the elastic component generally at the posterior region of said article is substantially greater than the elastic tension of the elastic component at the anterior region.

22. An absorbent article as set forth in claim 20 wherein the elastic tension of the elastic component generally at the posterior region of said article is substantially greater than the elastic tension of the elastic component at the central region.

23. An absorbent article as set forth in claim 19 wherein the elastic component further defines an inner boundary of the path along which the elastic component extends, said inner boundary being generally non-parallel to the outer boundary of said path.

24. An absorbent article as set forth in claim 19 wherein the elastic component further defines an inner boundary of the path along which the elastic component extends, said inner boundary being generally non-parallel to the longitudinal axis of the article and defining an inner curvature of the elastic component which is different from the outer curvature of said elastic component.

25. An absorbent article as set forth in claim 24 wherein the inner curvature of the elastic component is substantially less than the outer curvature of said elastic component.

26. An absorbent article as set forth in claim 19 wherein the outer curvature of the elastic component is at least about 1.25 inches.

27. A method for making an absorbent article having an anterior region, a posterior region and a central region interconnecting the anterior and posterior regions and extending longitudinally therebetween, said method comprising:
securing an elastic component to the absorbent article along a path which extends generally continuously from the anterior region of said article through the central region to the posterior region thereof, said elastic component defining a width of said path; and
removing at least a portion of the elastic component of said article to narrow the width of the path defined by the elastic component along at least a segment of the path to provide an elastic tension gradient along the length of the path.

28. A method as set forth in claim 27 wherein the path defined by the elastic component has a generally uniform width along the length of the path prior to the step of removing at least a portion of the elastic component.

29. A method as set forth in claim 27 wherein the elastic component is secured to the absorbent article along a path which is generally non-parallel to a longitudinal axis of the article whereby said elastic component has an initial outer curvature of the elastic component, the step of removing at least a portion of the elastic component forming a final outer curvature of the elastic component which is substantially greater than the initial outer curvature of the elastic component.
